SEQUENCE LISTING (22 sequences)

SEQ ID NO: 1 - RNA, 20 nt, synthetic construct
  source 1..20: mol_type = other RNA
tagatcctaacctagagccc

SEQ ID NO: 2 - RNA, 20 nt, synthetic construct
  source 1..20: mol_type = other RNA
tctcctatggcaggaagaag

SEQ ID NO: 3 - RNA, 20 nt, synthetic construct
  source 1..20: mol_type = other RNA
gaaggaatcgaagaagaagg

SEQ ID NO: 4 - RNA, 20 nt, synthetic construct
  source 1..20: mol_type = other RNA
gaaagaatcgaagaaggagg

SEQ ID NO: 5 - RNA, 20 nt, synthetic construct
  source 1..20: mol_type = other RNA
ccgattccttcgggcctgtc

SEQ ID NO: 6 - RNA, 20 nt, synthetic construct
  source 1..20: mol_type = other RNA
tctccgcttcttcctgccat

SEQ ID NO: 7 - RNA, 20 nt, synthetic construct
  source 1..20: mol_type = other RNA
gcttaggcatctcctatggc

SEQ ID NO: 8 - RNA, 20 nt, synthetic construct
  source 1..20: mol_type = other RNA
ggctctaggttaggatctac

SEQ ID NO: 9 - DNA, 20 bp, synthetic construct
  source 1..20: mol_type = other DNA
tagatcctaacctagagccc

SEQ ID NO: 10 - DNA, 20 bp, synthetic construct
  source 1..20: mol_type = other DNA
tctcctatggcaggaagaag

SEQ ID NO: 11 - DNA, 20 bp, synthetic construct
  source 1..20: mol_type = other DNA
gaaggaatcgaagaagaagg

SEQ ID NO: 12 - DNA, 20 bp, synthetic construct
  source 1..20: mol_type = other DNA
gaaagaatcgaagaaggagg

SEQ ID NO: 13 - DNA, 20 bp, synthetic construct
  source 1..20: mol_type = other DNA
ccgattccttcgggcctgtc

SEQ ID NO: 14 - DNA, 20 bp, synthetic construct
  source 1..20: mol_type = other DNA
tctccgcttcttcctgccat

SEQ ID NO: 15 - DNA, 20 bp, synthetic construct
  source 1..20: mol_type = other DNA
gcttaggcatctcctatggc

SEQ ID NO: 16 - DNA, 20 bp, synthetic construct
  source 1..20: mol_type = other DNA
ggctctaggttaggatctac

SEQ ID NO: 17 - RNA, 21 nt, synthetic construct
  source 1..21: mol_type = other RNA
gcagaactacacaccagggcc

SEQ ID NO: 18 - RNA, 21 nt, synthetic construct
  source 1..21: mol_type = other RNA
ggatagatgtaaaagacacca

SEQ ID NO: 19 - RNA, 21 nt, synthetic construct
  source 1..21: mol_type = other RNA
gcggcagcatagtgagcccag

SEQ ID NO: 20 - RNA, 20 nt, synthetic construct
  source 1..20: mol_type = other RNA
tcagtttacacccgatccac

SEQ ID NO: 21 - protein, 5 aa, synthetic construct
  SITE 1: note = D-Tyrosine
  SITE 2: note = Ornithine
  SITE 4: note = Beta-(2-Naphthyl)-Alanine
  SITE 1..5: note = Cyclic
YXRAG

SEQ ID NO: 22 - protein, 5 aa, synthetic construct
  SITE 1: note = Beta-(2-Naphthyl)-Alanine
  SITE 3: note = D-Tyrosine
  SITE 4: note = Ornithine
  SITE 1..5: note = Cyclic
AGYXR

(19) United States
(12) Patent Application Publication
(10) Pub. No.: US 2025/0360083 A1
(43) Pub. Date: Nov. 27, 2025

(54) LIPID NANOPARTICLE FORMULATIONS AND METHODS OF USE THEREOF

(71) Applicant: Board of Regents of the University of Nebraska, Lincoln, NE (US)

(72) Inventors: Howard E. Gendelman, Omaha, NE (US); Sudipta Panja, Omaha, NE (US); David Oupicky, Omaha, NE (US); Pallavi Deol, Omaha, NE (US); Lubaba Aria Zaman, Omaha, NE (US); Mahmudul Hasan, Omaha, NE (US); Milankumar Patel, Omaha, NE (US)

(21) Appl. No.: 19/113,841

(22) PCT Filed: Sep. 21, 2023
(86) PCT No.: PCT/US2023/074752

§ 371 Date: Mar. 20, 2025

Related U.S. Application Data
(60) Provisional application No. 63/376,507, filed on Sep. 21, 2022.
(60) Provisional application No. 63/530,317, filed on Aug. 2, 2023.

(51) Int. Cl. A61K 9/1271; A61K 9/51; A61K 47/54; A61K 47/69; B82Y 5/00; C12N 9/22; C12N 15/113; C12N 15/88

(52) U.S. Cl. CPC A61K 9/1271; A61K 9/5123; A61K 47/544; A61K 47/6935; C12N 9/222; C12N 15/1132; C12N 15/88; B82Y 5/00

(57) ABSTRACT

Disclosed herein are lipid nanoparticles comprising plurality of lipids, a targeting moiety for an HIV-1 chemokine receptor, and a CRISPR nucleic acid complementary to an HIV-1 gene, pharmaceutical compositions and methods of use thereof.

### CROSS REFERENCE

This application claims priority to U.S. Ser. No. 63/376,507, filed Sep. 21, 2022, and U.S. Ser. No. 63/530,317, filed Aug. 2, 2023, the contents of each of which are incorporated herein by reference in their entireties.

### STATEMENT AS TO FEDERALLY FUNDED RESEARCH

This invention was made with government support under Grants No. 5R01 MH121402, 5R01 MH115860, T32 NS105594, R01 MH121402. R01 A1158160, R01 DA054535, R01 NS126089, R01 A1145542, R01 NS036126. R01 MH115860, R33 DA041018, and R01 NS034239, all awarded by the National Institutes of Health. The government has certain rights in the invention.

### SUMMARY

Disclosed herein, in some embodiments, are lipid nanoparticles comprising a plurality of lipids, a CXCR4 targeting moiety, and a CRISPR nucleic acid complementary to a sequence within an HIV-1 gene. For example, in some embodiments, provided herein are lipid nanoparticles comprising (a) a shell comprising a plurality of lipids and having (i) an exterior surface comprising a CXCR4 targeting moiety linked to a PEG-lipid conjugate and (ii) an interior surface defining an inner cavity: and (b) a CRISPR nucleic acid complementary to a sequence within an HIV-1 gene encapsulated within the inner cavity of the shell. Additionally provided herein, in some embodiments, are pharmaceutical compositions comprising: (a) lipid nanoparticles comprising a plurality of lipids, a CXCR4 targeting moiety, and a CRISPR nucleic acid complementary to a sequence within an HIV-1 gene. and (b) a pharmaceutically acceptable excipient. In some embodiments, provided herein are pharmaceutical compositions comprising: (a) lipid nanoparticles comprising (i) a shell comprising a plurality of lipids and having an exterior surface comprising a CXCR4 targeting moiety linked to a PEG-lipid conjugate and an interior surface defining an inner cavity, and (ii) a CRISPR nucleic acid complementary to a sequence within an HIV-1 gene encapsulated within the inner cavity of the shell, and (b) a pharmaceutically acceptable excipient. Further provided herein, in some embodiments, are methods for the treatment and prevention of an HIV-1 infection in an individual in need thereof, comprising administering to the individual lipid nanoparticles disclosed herein or pharmaceutical compositions comprising said lipid nanoparticles and a pharmaceutically acceptable excipient. In some embodiments, provided herein are methods for the treatment and prevention of an HIV-1 infection in an individual in need thereof, comprising administering to the individual lipid nanoparticles comprising (a) a shell comprising a plurality of lipids and having (i) an exterior surface comprising a CXCR4 targeting moiety linked to a PEG-lipid conjugate and (ii) an interior surface defining an inner cavity; and (b) a CRISPR nucleic acid complementary to a sequence within an HIV-1 gene encapsulated within the inner cavity of the shell, or the pharmaceutical compositions comprising said lipid nanoparticles and a pharmaceutically acceptable excipient.

In some embodiments, the lipid nanoparticle comprises cationic lipids, zwitterionic lipids, sterol, and PEG-lipid conjugates. In some embodiments, the lipid nanoparticle comprises C12-200, DOPE, β-sitosterol, and DMG-PEG. In some embodiments, the lipid nanoparticle comprises MC3, DSPC, β-sitosterol, DOPS, and DMG-PEG. In some embodiments, the CXCR4 targeting moiety is a CXCR4 inhibitor. In some embodiments, the CXCR4 targeting moiety is AMD070, or a pharmaceutically acceptable salt thereof. In some embodiments, the CXCR4 targeting moiety is a cyclic polypeptide having the sequence D-Tyr-Orn-Arg-Nal-Gly-conjugated to DSPE-PEG.

Also provided herein, in some embodiments, is a pharmaceutical composition, comprising: (a) the lipid nanoparticle disclosed herein, and (b) a pharmaceutically acceptable excipient.

Also provided herein, in some embodiments, is a method of disrupting the transcription of an exon of an HIV-1 sequence in an individual in need thereof, comprising administering to the individual a lipid nanoparticle disclosed herein or a pharmaceutical composition disclosed herein.

Also provided herein, in some embodiments, is a method of excising all or a portion of an HIV-1 sequence in an individual in need thereof, comprising administering to the individual a lipid nanoparticle disclosed herein or a pharmaceutical composition disclosed herein.

Also provided herein, in some embodiments, is a method of treating an HIV-1 infection in an individual in need thereof, comprising administering to the individual a lipid nanoparticle disclosed herein or a pharmaceutical composition disclosed herein.

Also provided herein, in some embodiments, is a method of preventing an HIV-1 infection in an individual in need thereof, comprising prophylactically administering to the individual a lipid nanoparticle disclosed herein or a pharmaceutical composition disclosed herein.

Also provided herein is a method of preventing transmission of an HIV-1 virus from a first individual to a second individual, comprising administering to the first individual a lipid nanoparticle disclosed herein is a pharmaceutical composition disclosed herein.

Other objects and advantages will become apparent to those skilled in the art from a consideration of the ensuing Detailed Description, Examples, and Claims.

### BRIEF DESCRIPTION OF THE DRAWINGS

FIG. 1A illustrates a synthetic scheme for DSPE-PEG-AMD070 conjugate and lipid nanoparticles.

FIG. 1B is an ¹H NMR spectrum of the DSPE-PEG-AMD070 in CDCl₃.

FIG. 2 illustrates a flow cytometric analysis of CXCR4 receptor on JLat8.4 and U1 cells. U1 and JLat8.4 cells were cultured for three passages and then stained Live/Dead Fixable Far Red & Mouse anti-Human CXCR4-Alexa 488 antibody. Unstained cells for each channel (Flow minus one controls) were used to draw the flow gates. Live singlet CXCR4+ cells (Red) are presented in histogram with unstained cells (Blue) as control.

FIG. 3 illustrates the cell viability analysis of T-LNP and C-LNP against J-Lat8.4 and U1 cells. The viability assay was performed using CTB assay at 40 h post-incubation with LNPs. Both the J-Lat8.4 and U1 cells showed viability above 80% at a dose as high as 0.16 μg/million cells. The error bars represent mean ± standard error of mean from six biological replicates.

FIG. 4 illustrates the dose-dependent luciferase expression in J-Lat8.4 and U1 cells at 40 h post-treatment with T-LNP and C-LNP. T-LNPs showed a more than 2-fold increase in luciferase expression in J-Lat8.4 cells at 0.16 μg/million cells. In U1 cells, T-LNPs showed a similar level of luciferase expression as C-LNPs. Statistical analysis was performed using two-way ANOVA. ****, P<0.0001 and ns=not significant.

FIG. 5 illustrates the effect of a CXCR4 inhibitor (AMD070) on LNP-mediated luciferase expression in J-Lat8.4 cells. LNP at 0.16 μg/million cells (equivalent to mRNA) dose was used to treat the cells preincubated with CXCR4 inhibitor (6.6 μM) over 2 h. Cells without inhibitor served as control for both types of LNPs. Statistical analysis was performed using two-way ANOVA. **, P<0.01; and ns=not significant.

FIG. 6A illustrates chemical structures of MC3 and C12-200 ionizable lipids.

FIG. 6B is a pie chart representing the lipid components used to formulate exemplary lipid nanoparticles.

FIG. 6C is an exemplary gel image displaying intact proviral DNA band (3 kb) and the excised band (428 bp)

FIG. 6D illustrates an excised band intensity on the gel blot.

FIG. 6E is a schematic presentation of the synthesis of DSPE-PEG-CycPep by activated acid-amine coupling reaction.

FIG. 7 is an ¹H NMR spectrum of the DSPE-PEG-AMD070 obtained by dissolving in CDCl₃. The NMR signals corresponding to DSPE, PEG and AMD070 protons are designated by a and b, c, d. respectively.

FIG. 8A and FIG. 8B are pie charts of lipid components (% mol) used to formulate T-LNP and C-LNP, respectively. FIG. 8C and FIG. 8D illustrate size and zeta potential measured by dynamic light scattering and mRNA encapsulation efficiency (EE) of T-LNP and C-LNP, respectively. FIG. 8E and FIG. 8F are cryo-EM images of T-LNP and C-LNP, respectively.

FIG. 9A and FIG. 9B illustrate 6-(p-Toluidino)-2-naphthalenesulfonyl chloride (TNS) assay results used to determine the pKa of C-LNP and T-LNP, respectively. Both the LNPs showed pKa between pH 6.20 to 6.37 suitable for endosomal escape.

FIG. 10A and FIG. 10B illustrate cell viability of J-Lat8.4, Jurkat E6 and U1 cells after treatment with T-LNP and C-LNP, respectively. Cell viability assay was performed using Cell Titer Blue assay 72 h post-incubation with LNPs. All cell lines showed viability above 70% at a dose as high as 1 μg/million cells.

FIG. 11A and FIG. 11B demonstrate the effect of CXCR4 inhibitor (AMD070) on LNP-mediated luciferase expression in U1, J-Lat8.4 and JE6 cell lines for T-LNP and C-LNP, respectively. Unlike C-LNP, T-LNP showed CXCR4-dependent luciferase expression. LNP at an mRNA dose of 1 μg/million cells was used to treat the cells preincubated for 30 min with CXCR4 inhibitor, AMD070, at doses 6.25, 12.5, and 25 μM. The treatment group without AMD070 served as the control.

FIG. 12A shows a gel image of results from DNA extracted from U1, JLat 8.4 and Jurkat E6 cell lines 72 hours after treatment with LNPs at a dose of 1 μg/million cells. The gel image displays an intact proviral DNA band at 3 kb along with the excised band at 428 bp. FIG. 12B shows a schematic representation of the level of CXCR4 expression in U1 (28%), Jlat (99%), and JE6 (99%) cell lines. FIG. 12C shows an excised band intensity on the gel. The band intensity was quantified by using ImageJ software. Unlike C-LNP, T-LNP showed CXCR4-expressing cell-specific viral DNA excision.

FIG. 13A shows an analysis of the gel image of T-LNP treated cells showed a reduction of viral DNA excision after preincubation with CXCR4 blocker, AMD070 (at 50 μM). FIG. 13B shows a plot of band intensity. ImageJ software quantified the band intensity and plotted using GraphPad Prism 10.0.0.

FIG. 14A and FIG. 14B show the biodistribution of LNPs was visualized under IVIS at 6 h post-injection (through tail vein) of T-LNP (FIG. 14A) and T-LNP-DOPS (FIG. 14B) (loaded with FLuc mRNA), respectively. A combination of active and endogenous targeting results in lymphoid organs' specific mRNA expression of T-LNP-DOPS in 8 weeks old BALB/cJ mice.

### DETAILED DESCRIPTION

Disclosed herein, in some embodiments, are lipid nanoparticles comprising a plurality of lipids, a CXCR4 targeting moiety, and a CRISPR nucleic acid complementary to a sequence within an HIV-1 gene. In some embodiments, provided herein is a lipid nanoparticle, comprising: (a) a surface comprising a plurality of lipids and CXCR4 targeting moiety linked to a PEG-lipid conjugate; and (b) a CRISPR nucleic acid complementary to a sequence within an HIV-1 gene encapsulated within the lipid nanoparticle. In some embodiments, provided herein is a lipid nanoparticle comprising (a) a shell comprising a plurality of lipids and having (i) an exterior surface comprising a CXCR4 targeting moiety linked to a PEG-lipid conjugate and (ii) an interior surface defining an inner cavity; and (b) a CRISPR nucleic acid complementary to a sequence within an HIV-1 gene encapsulated within the inner cavity of the shell. Additionally provided herein, in some embodiments, are pharmaceutical compositions comprising lipid nanoparticles disclosed herein, and a pharmaceutically acceptable excipient. Further provided herein, in some embodiments, are methods for the treatment and prevention of an HIV-1 infection in an individual in need thereof, comprising administering to the individual lipid nanoparticles disclosed herein or pharmaceutical compositions comprising said lipid nanoparticles and a pharmaceutically acceptable excipient.

### Definitions

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method. compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate. cyclopentanepropionate, digluconate, dodecylsulfate, ethanesulfonate, formate, fumarate, glucoheptonate, glycerophosphate, gluconate, hemisulfate. heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%. etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human.” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

The term “lipid nanoparticles” as used herein, refers to nanoparticles comprising a core and a shell where the core is enclosed by a shell comprising one or more lipids.

The term “shell” as used herein, refers to the outer portion of the lipid nanoparticle and is typically comprised of different components than the core. The shell is characterized as having an exterior surface, which does not face or is not in contact with the core of the lipid nanoparticle, and an interior surface, which faces or is in contact with the core of the lipid nanoparticle and defines the inner cavity of the lipid nanoparticle. For example, the lipid nanoparticles disclosed herein comprise a shell formed by one or more lipids (such as zwitterionic lipids, cationic lipids, and PEG-lipid conjugates) and is characterized as having an exterior surface comprising a targeting moiety (e.g., a targeting moiety linked to a PEG-lipid conjugate forming part of the shell) and an interior surface that defines the inner cavity of the lipid nanoparticle.

The term “core” as used herein, refers to the internal portion of the lipid nanoparticle that is enclosed by the shell and is in contact with the interior surface of said shell. The core is typically comprised of different components than the shell. For example, the lipid nanoparticles disclosed herein comprise a core containing one or more nucleic acids (e.g., a CRISPR nucleic acid complementary to a sequence within an HIV-1 gene) and one or more lipids. In some embodiments, the nucleic acid is encapsulated by one or more lipids (such as zwitterionic lipids and cationic lipids).

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, the term “crRNA” means a non-coding short RNA sequence which bind to a complementary target DNA sequence. The crRNA sequence binds to a Cas enzyme (e.g., Cas9) and the crRNA sequence guides the complex via pairing to a specific target DNA sequence.

As used herein, the term “tracrRNA” or trans-activating CRISPR RNA means an RNA sequence that base pairs with the crRNA (to form a functional guide RNA (gRNA)). The tracrRNA sequence binds to a Cas enzyme (e.g., Cas9), while the crRNA sequence of the gRNA directs the complex to a target sequence.

As used herein, the term “gRNA” means the crRNA and a tracrRNA bound together. The gRNA binds to a Cas enzyme (e.g., Cas9) and guides the Cas enzyme to the target sequence.

As used herein, the term “sgRNA” means a single RNA construct comprising a crRNA sequence and a tracrRNA sequence.

As used herein, the term “mosaic crRNAs” mean crRNAs that are constructed from a multiple sequence alignment of separate viral strains, for example separate HIV-1 strains (92UG_029, KER2008, 99KE_KNH1135 etc.) or HIV-2 strains.

As used herein, the term “overlapping sequence” or “overlapping exon” means exons or genes that are transcribed in different reading frame from the same part of the DNA sequence.

### Lipid Nanoparticles (LNPs)

Provided herein, in some embodiments, are lipid nanoparticles that are selective for CD4+ T cells and/or myeloid cells known to host latent HIV proviral DNA. Thus, the lipid nanoparticles provided herein, in some embodiments, improve the delivery of CRISPR-Cas9 therapies to HIV infected CD4+ T cells and/or myeloid cells, facilitate excising of HIV genome in an HIV infected cell and help with viral elimination. Disclosed herein, in some embodiments, are lipid nanoparticles with efficient mRNA-delivering abilities that are intended to exhibit low toxicity and reduced immunogenicity for delivery of CRISPR-Cas9 therapies.

In some embodiments, provided herein are lipid nanoparticles comprising a plurality of lipids, a targeting moiety for an HIV-1 chemokine receptor, and a CRISPR nucleic acid complementary to a sequence within an HIV-1 gene. In some embodiments, the HIV-1 chemokine receptor is CXCR4. The targeted lipid nanoparticles disclosed herein exhibit higher mRNA delivery efficiency than conventional LNPs which are nonspecifically targeted.

In some embodiments, provided herein is a lipid nanoparticle, comprising a plurality of lipids, a CXCR4 targeting moiety, and a CRISPR nucleic acid complementary to a sequence within an HIV-1 gene.

In some embodiments, provided herein is a lipid nanoparticle, comprising: (a) a surface comprising a plurality of lipids and CXCR4 targeting moiety linked to a PEG-lipid conjugate; and (b) a CRISPR nucleic acid complementary to a sequence within an HIV-1 gene encapsulated within the lipid nanoparticle.

In some embodiments, the lipid nanoparticles described herein encapsulate and deliver a CRISPR nucleic acid complementary to a sequence within an HIV-1 gene.

In some embodiments, the lipid nanoparticles are formed using a variety of lipids including, but are not limited to, cationic lipids, anionic lipids, zwitterionic (neutral) lipids, non-polar lipids, sterols, and lipids modified by other agents or compounds or conjugated or linked to other agents or compounds including, but not limited, to polymers, or a combination thereof, such as PEG-lipid conjugates, and spleen-targeting helper lipids.

Examples of lipids used to produce lipid nanoparticles include, but are not limited to, DOTMA (1,2-di-O-octadecenyl-3-trimethylammonium propane), DOSPA (N-(1-(2,3-dioleyloxy)propyl)-N-2-(sperminecarboxamido)ethyl)-N,N-dimethylammonium trifluoracetate), DOTAP (1,2-dioleoyl-3-trimethylammonium propane), DMRIE (N-(1,2-dimyristyloxyprop-3-yl)-N,N-dimethyl-N-hydroxyethyl ammonium), DC-cholesterol (3β-(N-(N′,N′-dimethylaminoethane)-carbamoyl) cholesterol), DOTAP-cholesterol (1,2-dioleoyl-3-trimethylammonium propane;(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]lphenanthren-3-ol), GAP-DMORIE-DPyPE (Vaxfectin; (±)-N-(3-aminopropyl) -N,N-dimethyl-2,3-bis(cis-9)-tetradeceneyloxy)-1-propanaminium;1,2-diphytanoyl-sn-glycero-3-phosphoethanolamine), and GL67A (GL67-DOPE-DMPE-polyethylene glycol (PEG) (cholest-5-en-3-ol (3β)-,3-[(3-aminopropyl)[4-[(3-aminopropyl)amino]butyl]carbamate;1,2-dileoyl-sn-3-phosphoethanolamine;dimyristoylphosphoethanolamine; PEG), and pharmaceutically acceptable salts thereof.

Cationic lipids include, but are not limited to, 1,2-di-O-octadecenyl-3-trimethylammonium propane (DOTMA), N,N-dioleyl-N,N-dimethylammonium chloride (DODAC), didodecyldimethylammonium bromide (DDAB), N,N-dimethyl2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP). 1,2-Dilinoleyoxy-3-(dimethylamino)acetoxypropane (DLinDAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoley loxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), 3-(N,N-Dilinoleylamino)-1,2-propanediol (DLinAP), 3-(N,N-Diolcylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), (3aR,5s,6aS)-N,N-dimethyl-2,2-di((9Z,12Z)-octadeca-9,12-dienyl)tetrahydro-3aH-cyclopenta[d][1,3]dioxol-5-amine (ALNY-100), DODAP (1,2-dioleoyl-3-dimethylammonium propane), GL67 (cholest-5-en-3-ol (βB)-,3-[(3-aminopropyl)[4-[(3-aminopropyl)amino]butyl]carbamate), ethyl PC, DOSPA (N-(1-(2,3-dioleyloxy)propyl)-N-2-(sperminecarboxamido)ethyl)-N,N-dimethylammonium trifluoracetate), DOGS (dioctadecylamidoglycyl carboxyspermine), DORIE (N-(2-hydroxyethyl)-N,N-dimethyl-2,3-bis(((Z)-octadec-9-en-1-yl) oxy)propan-1-aminium), DMRIE (N-(1,2-dimyristyloxyprop-3-yl)-N,N-dimethyl-N-hydroxyethyl ammonium), GAP-DLRIE ((+/−)-N-(3-aminopropyl)-N,N-dimethyl-2,3-bis(dodecyloxy)-1-propanaminium), diC14-amidine, 3β-[N-(N′,N′-dimethylaminoethane)-carbamoyl]cholesterol (DC-Chol), dimethyldioctadecylammonium (DDA), 1,2-dioleoyl-3-trimethylammonium propane (DOTAP), 1,2-dimyristoyl-3-trimethylammonium-propane (DMTAP), 1,2-stearoyl-3-trimethylammonium-propane (DSTAP) and N-(4-carboxybenzyl)-N,N-dimethyl-2,3-bis(oleoyloxy)propan-1-aminium (DOBAQ), egg phosphatidylcholine, and cholesterol-polyethylene glycol, 98N12-5 (isomer of triethylenetetramine-laurylaminopropionate with a free internal amine, cholesterol, and mPEG2000-C14 glyceride), C12-200 (CAS #: 1220890-25-4; 1,1-((2-(4-(2-((2-(bis(2-hydroxydodecyl)amino)ethyl)(2-hydroxydodecyl) amino)ethyl)piperazin-1-yl)ethyl)azanediyl)bis(dodecan-2-ol)), DLin-KC2-DMA (KC2) (CAS #: 1190197-97-7; 2,2-dilinoleyl-4-(2-dimethylaminoethyl)-[1,3]-dioxolane), DLin-MC3-DMA (MC3) (CAS #: 1224606-06-7; dilinoleylmethyl-4-dimethylaminobutyrate), XTC (2,2-dilinoleyl-4-dimethylaminoethyl-[1,3]-dioxolane), MD1 (CKK-E12; 3,6-bis({4-[bis(2-hydroxydodecyl)amino]butyl})piperazine-2,5-dione), 7C1 (C15 epoxide-terminated lipid), and pharmaceutically acceptable salts thereof.

Examples of zwitterionic (neutral) lipids include, but are not limited to, DSPC (distearoylphosphatidylcholine), dioleoylphosphatidylcholine (DOPC), dioleoylphosphatidylglycerol (DOPG), dioleoyl-phosphatidylethanolamine 4-(N-maleimidomethyl)-cyclohexane-1-carboxy late (DOPE-mal), dipalmitoylphosphatidylglycerol (DPPG), palmitoyloleoylphosphatidylethanolamine (POPE), dipalmitoyl phosphatidyl ethanolamine (DPPE), dimyristoy lphosphoethanolamine (DMPE), 16-O-monomethyl PE, 16-O-dimethyl PE, 18-1-trans PE, 1-stearoyl-2-oleoyl-phosphatidyethanolamine (SOPE), DPSC (distearoylphosphatidylcholine), DPPC (dipalmitoylphosphatidylcholine), POPC (palmitoyloleoylphosphatidylcholine), DOPE (1,2-dileoyl-sn-3-phosphoethanolamine), DSPE (1,2-distearoyl-sn-glycero-3-phosphoethanolamine), DMG (dimyristoyl glycerol), phosphatidylserines, phosphatidylethanolamines, phosphatidylcholines, sphingomyelins, sphingophospholipids, betaine lipids (e.g. lauramidopropyl betaine), and SM (sphingomyelin).

Anionic lipids include, but are not limited to, phosphatidylglycerols (PG), phosphatidic acid and phosphatidylinositol phosphates. Non-polar lipids may include but are not limited to glycerides (mono, di, and triglycerides) and other non-charged lipids.

In some embodiments, the lipids are modified or conjugated to other molecules (e.g., chemically linked such as by an acid-amine coupling reaction between an available acid (or amine) on the lipid and an available amine (or acid) available on the other molecule(s) to produce a conjugate). In some embodiments, the lipid is conjugated to a polymer. In some embodiments, the polymer is polyethylene glycol (PEG). In some embodiments, the PEG has a molecular weight from about 200 g/mol to 10,000 g/mol. In some embodiments. the PEG has a molecular weight from about 200 g/mol to 1,000 g/mol. In some embodiments, the PEG has a molecular weight from about 200 g/mol to 800 g/mol. In some embodiments, the PEG is any molecular weight form of PEG including but not limited to PEG₂₀₀, PEG₃₀₀, PEG₄₀₀, PEG₆₀₀, PEG₁₀₀₀, PEG₂₀₀₀, PEG₃₀₀₀, PEG₆₀₀₀, and PEG₈₀₀₀. Examples of PEG-lipid conjugates include, but are not limited to, DMG-PEG, DSPE-PEG, and DMP-PEG.

In some embodiments, the lipid nanoparticles further comprise a sterol. Exemplary sterols include, but are not limited to, β-sitosterol and cholesterol. In some embodiments, the sterol is a phytosterol.

In some embodiments, the lipid nanoparticles further comprise a spleen targeting helper lipid. Spleen targeting helper lipids include but are not limited to 1,2-dioleoyl-sn-glycero-3-phospho-L-serine (DOPS), phosphatidylserine (PS), DOTAP, and DOPE.

In some embodiments, the lipid nanoparticle comprises cationic lipids, zwitterionic lipids, sterol, and PEG-lipid conjugates.

In some embodiments, the lipid nanoparticle comprises C12-200, DOPE, β-sitosterol, and DMG-PEG.

In some embodiments, the lipid nanoparticle comprises cationic lipids, zwitterionic lipids, sterol, PEG-lipid conjugates, and a spleen targeting helper lipid.

In some embodiments, the lipid nanoparticle comprises MC3, DSPC, β-sitosterol, DOPS, and DMG-PEG.

In some embodiments, the CXCR4 targeting moiety is a CXCR4 inhibitor. In some embodiments, the CXCR4 targeting moiety is a CXCR4 antagonist.

In some embodiments, the CXCR4 targeting moiety is linked to a lipid, such as a PEG-lipid conjugate. In some embodiments, the CXCR4 targeting moiety is covalently linked to a lipid such as a PEG-lipid conjugate.

Exemplary CXCR4 targeting moieties include, but are not limited to: plerixafor (AMD3100), having the structure:

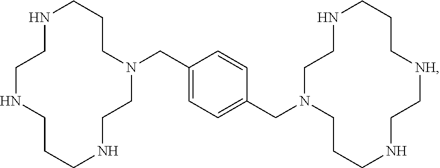 [SMILES: c1cc(CN2CCCNCCNCCCNCC2)ccc1CN1CCCNCCNCCCNCC1]

or a pharmaceutically acceptable salt thereof;
AMD3465, having the structure:

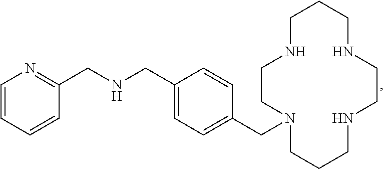 [SMILES: c1ccc(CNCc2ccc(CN3CCCNCCNCCCNCC3)cc2)nc1]

or a pharmaceutically acceptable salt thereof;
IT1t, having the structure:

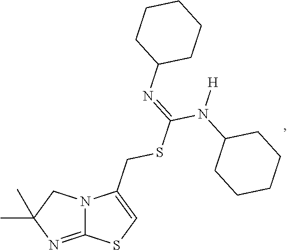 [SMILES: CC1(C)CN2C(CS/C(=N/C3CCCCC3)NC3CCCCC3)=CSC2=N1]

or a pharmaceutically acceptable salt thereof;
KRH-3955, having the structure:

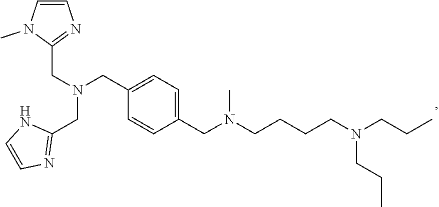 [SMILES: CCCN(CCC)CCCCN(C)Cc1ccc(CN(Cc2ncc[nH]2)Cc2nccn2C)cc1]

or a pharmaceutically acceptable salt thereof;
AMD070, having the structure:

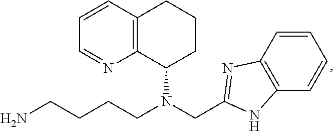 [SMILES: NCCCCN(Cc1nc2ccccc2[nH]1)[C@H]1CCCc2cccnc21]

or a pharmaceutically acceptable salt thereof;
HF51116, having the structure:

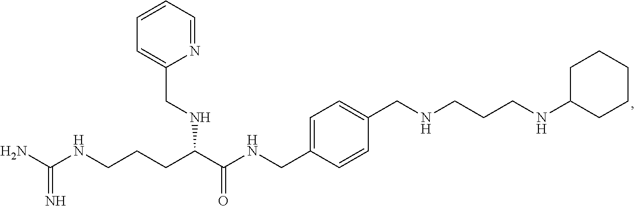 [SMILES: N=C(N)NCCC[C@H](NCc1ccccn1)C(=O)NCc1ccc(CNCCCNC2CCCCC2)cc1]

or a pharmaceutically acceptable salt thereof;
BPRCX807, having the structure:

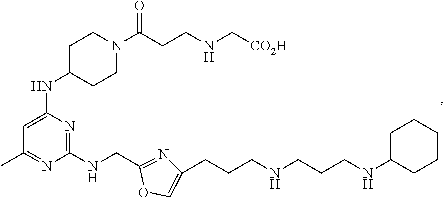 [SMILES: Cc1cc(NC2CCN(C(=O)CCNCC(=O)O)CC2)nc(NCc2nc(CCCNCCCNC3CCCCC3)co2)n1]

or a pharmaceutically acceptable salt thereof; and a customized cyclic polypeptide having the sequence (cyclo-[Nal-Gly-(D-Tyr)-Orn-Arg]) (also called CycPep) and structure:

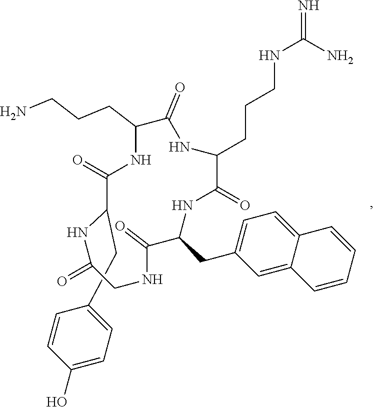 [SMILES: N=C(N)NCCCC1NC(=O)C(CCCN)NC(=O)C(Cc2ccc(O)cc2)NC(=O)CNC(=O)[C@H](Cc2ccc3ccccc3c2)NC1=O]

or a pharmaceutically acceptable salt thereof.

In some embodiments, the CXCR4 targeting moiety is linked to PEG-lipid conjugates. In some embodiments, the PEG-lipid conjugates are DSPE-PEG. In some embodiments the CXCR4 targeting moiety is CycPep conjugated to DSPE-PEG.

In some embodiments, the lipid nanoparticle comprises MC3, DSPC, β-sitosterol, DOPS, DMG-PEG, and CycPep-DSPE-PEG (CycPep conjugated to DSPE-PEG).

### crRNAs (CRISPR RNAs)

Disclosed herein, in some embodiments, are lipid nanoparticles comprising nucleic acids encoding for mosaic crNRA sequences for the treatment and prevention of HIV infections. In some embodiments, the crRNA sequences bind to a DNA sequence within an HIV genome (e.g., HIV-1 or HIV-2). In some embodiments, the lipid nanoparticles described herein package or encapsulate crRNAs. In some embodiments, the crRNA bind to sequences within an HIV gene including, but not limited to tat, rev, env-gp41, gag-p1, gag-p6, vif, vpr, vpu, and nef. In some embodiments the crRNAs bind to overlapping exons in two or more of these genes.

In some embodiments, the crRNAs are “mosaic crRNAs.” In some embodiments, the mosaic crRNA is constructed from a multiple sequence alignment of separate HIV viral strains, for example separate HIV-1 or HIV-2 strains. In some embodiments, the target sequence of the mosaic crRNA is a theoretical composite of an HIV-1 or HIV-2 DNA sequences, for example sequences that retain a high (≥50%) or low (<50%) levels of conservation across isolated HIV strains.

The 10 kilobase pair (kb) genome of HIV-1 encodes 3 structural (gag, pol, and env) polyproteins and 6 non-structural (tat, rev, vif, vpu, vpr, and nef) proteins from 3 overlapping alternate reading frames. HIV-1 has four groups. Group M (Major) accounts for a majority of all HIV-1 cases. HIV-1, group M has nine named strains: A, B, C, D, F, G, H, J, and K. Additionally, Different subtypes can combine genetic material to form a hybrid virus, known as a ‘circulating recombinant form’ (CRFs). HIV-1, group M, strain B strain is the most common strain of HIV in the U.S. Worldwide, the most common HIV strain is HIV-1, group M, strain C. HIV-1 has three additional groups-groups N, O, and P.

In some embodiments, a mosaic crRNA is constructed from a multiple sequence alignment of two or more HIV-1, group M strains selected from: A, B, C, D, F, G, H, J, and K.

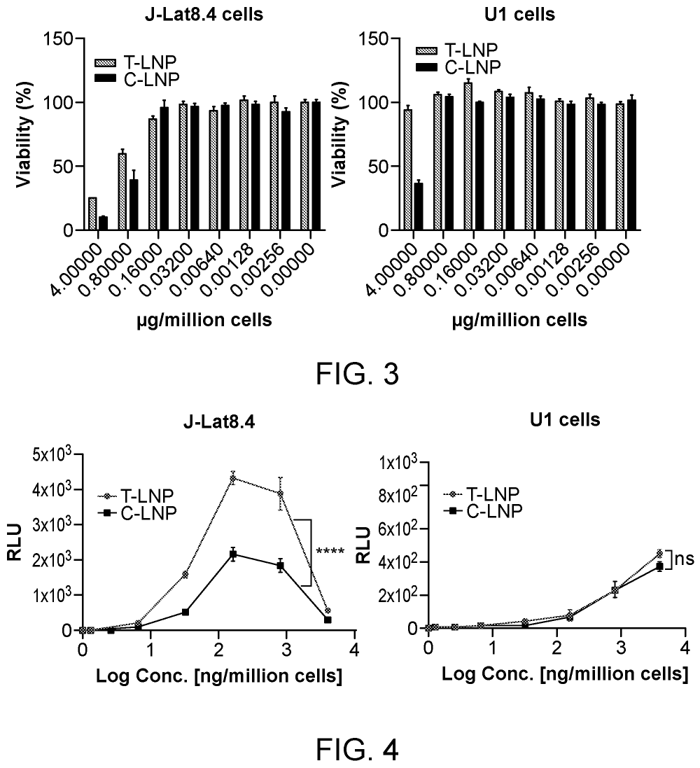

To construct mosaic crRNAs, a consensus HIV sequence can be created. The consensus sequence is based on the most recent alignment for the fullest spectrum of HIV-1 sequences, for example using the Los Alamos National Laboratory database for HIV sequence (hiv.lanl.gov). The Los Alamos database contains 4004 variant sequences. FIG. 3 summarizes the tat locus of all the 4004 sequences; the height of the letters corresponds to percentage of sequences that has that nucleotide in that specific location. For example, the first position in FIG. 3 (location 5831 in the HXB2 reference genome) is an A—most of the sequences of the 4004 variants at location 5831 had an A. From all available sequences, a consensus sequence can be generated. Each nucleotide of the consensus sequence can be determined based on being present on most of the sequences, for example is at least 50% of sequences.

In some embodiments, a mosaic crRNA disclosed herein binds to a plurality of nucleic acids within an HIV-1 gene selected from the group consisting of: tat, rev, env-gp41, gag-p1, gag-p6, vif, vpr, vpu, and nef. In some embodiments, a mosaic crRNA disclosed herein binds to a plurality of nucleic acids of a gene encoding an HIV-1 protein selected from the group consisting of: Tat, Rev, Env-gp41, Gag-p1, Gag-p6, Vif, Vpr, Vpu, and Nef.

In some embodiments, a mosaic crRNA disclosed herein targets a consensus sequence derived from over 4000 HIV strains in a non-structural multiexon region. In some embodiments, the mosaic crRNA sequence is adjacent to an appropriate PAM sequence. In some embodiments, the mosaic crRNA sequence is adjacent to a S. pyogenes (spCas9) PAM sequence (NGG). In some embodiments, the mosaic crRNA sequence is adjacent to a S. aureus Cas9 (saCas9) PAM sequence (NNGRRT or NGRRN). PAMs for various Cas enzymes are described in Table 1 below, where “N” can be any nucleotide base.

TABLE 1

| CRISPR | | |
|---|---|---|
| Nucleases | Organism Isolated From | PAM Sequence (5′ to 3′) |
| SpCas9 | Streptococcus pyogenes | NGG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| LbCpf1 | Lachnospiraceae bacterium | TTTV |
| AsCpf1 | Acidaminococcus sp. | TTTV |

Advantages of the mosaic multiexon cleavage strategy are threefold. (1) First, mosaic crRNAs targeting multiexon regions superiorly reduce viral replication compared to crRNAs targeting LTR promoter DNA and single gene encoding proviral DNA as a result of CRISPR-Cas9 cleavage rather than excision. (2) Second, mosaic crRNAs retain broader coverage against transmitted founder HIV-1 strains compared to conventional CRISPR-Cas9 crRNAs designed against routinely tested laboratory strains of HIV. (3) Third, crRNAs targeting multiexon or regulatory regions display lower likelihood of generating CRISPR-resistant escape mutants.

In some embodiments, a mosaic crRNA disclosed herein binds to a plurality of nucleic acids within an overlapping exon. In some embodiments, the overlapping exon is part of a nucleic acid sequence of at least two HIV-1 genes selected from the group consisting of: tat, rev, env-gp41, gag-p1, gag-p6, vif, vpr, vpu, and nef. In some embodiments, the overlapping exon is part of a nucleic acid sequence of at least three HIV-1 genes selected from the group consisting of: tat, rev, env-gp41, gag-p1, gag-p6, vif, vpr, vpu, and nef. In some embodiments, the overlapping exon is part of a nucleic acid sequence of HIV-1 genes tat, rev, and env.

In some embodiments, a mosaic crRNA disclosed herein binds to a plurality of nucleic acids within an HIV-1 sequence (HXB2, complete genome; HIV1/HTLV-III/LAV reference genome; GenBank: K03455.1) selected from: tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

In some embodiments, the mosaic crRNA is selected from a crRNA of Table 2 below:

TABLE 2

| | Target DNA | |
|---|---|---|
| | Complementary | crRNA Seed |
| crRNA | Sequence | Sequence |
| Name | (5′ → 3′) | (5′ → 3′) |
| TatA₂ | TAGATCCTAA | UAGAUCCUAACCUAGAGCCC |
| | CCTAGAGCCC | (SEQ ID NO. 1) |
| TatD | TCTCCTATGG | UCUCCUAUGGCAGGAAGAAG |
| | CAGGAAGAAG | (SEQ ID NO: 2) |
| TatE | GAAGGAATCG | GAAGGAAUCGAAGAAGAAGG |
| | AAGAAGAAGG | (SEQ ID NO: 3) |
| TatE₂ | GAAAGAATCG | GAAAGAAUCGAAGAAGGAGG |
| | AAGAAGGAGG | (SEQ ID NO: 4) |
| TatF | CCGATTCCTT | CCGAUUCCUUCGGGCCUGUC |
| | CGGGCCTGTC | (SEQ ID NO: 5) |
| TatG | TCTCCGCTTC | UCUCCGCUUCUUCCUGCCAU |
| | TTCCTGCCAT | (SEQ ID NO: 6) |
| TatH | GCTTAGGCAT | GCUUAGGCAUCUCCUAUGGC |
| | CTCCTATGGC | (SEQ ID NO: 7) |
| TatI | GGCTCTAGGT | GGCUCUAGGUUAGGAUCUAC |
| | TAGGATCTAC | (SEQ ID NO: 8) |

In some embodiments, the mosaic crRNA is TatA2-UAGAUCCUAACCUAGAGCCC (SEQ ID NO. 1). In some embodiments, the mosaic crRNA is TatD-UCUCCUAUGGCAGGAAGAAG (SEQ ID NO: 2). In some embodiments, the mosaic crRNA is TatE-GAAGGAAUCGAAGAAGAAGG (SEQ ID NO: 3). In some embodiments, the mosaic crRNA is TatE2-GAAAGAAUCGAAGAAGGAGG (SEQ ID NO: 4). In some embodiments, the mosaic crRNA is TatF-CCGAUUCCUUCGGGCCUGUC (SEQ ID NO: 5). In some embodiments, the mosaic crRNA is TatG-UCUCCGCUUCUUCCUGCCAU (SEQ ID NO: 6). In some embodiments, the mosaic crRNA is TatH-GCUUAGGCAUCUCCUAUGGC (SEQ ID NO: 7). In some embodiments, the mosaic crRNA is Tatl-GGCUCUAGGUUAGGAUCUAC (SEQ ID NO: 8).

In some embodiments. a crRNA described herein targets one or more HIV genes selected from the group consisting of: LTR, CCR5, or gagD, or a combination thereof. Guide RNAs which target LTR1, gagD, and CCR5 include but are not limited to:

LTR1:
5′-GCAGAACTACACACCAGGGCC-3′;
gagD:
5′-GGATAGATGTAAAAGACACCA-3′;
CCR5 A:
5′-GCGGCAGCATAGTGAGCCCAG-3′;
CCR5 B:
5′-TCAGTTTACACCCGATCCAC-3′

In some embodiments, the cRNA is 80%, 85%, 90%, or 95% identical to a sequence provided herein.

In some embodiments, the mosaic crRNA is 80%, 85%, 90%, or 95% identical to TatA2-UAGAUCCUAACCUAGAGCCC (SEQ ID NO. 1). In some embodiments, the mosaic crRNA is 80%, 85%, 90%, or 95% identical to TatD-UCUCCUAUGGCAGGAAGAAG (SEQ ID NO: 2). In some embodiments, the mosaic crRNA is 80%, 85%, 90%, or 95% identical to TatE-GAAGGAAUCGAAGAAGAAGG (SEQ ID NO: 3). In some embodiments, the mosaic crRNA is 80%, 85%, 90%, or 95% identical to TatE2-GAAAGAAUCGAAGAAGGAGG (SEQ ID NO: 4). In some embodiments, the mosaic crRNA is 80%, 85%, 90%, or 95% identical to TatF-CCGAUUCCUUCGGGCCUGUC (SEQ ID NO: 5). In some embodiments. the mosaic crRNA is 80%, 85%, 90%, or 95% identical to TatG-UCUCCGCUUCUUCCUGCCAU (SEQ ID NO: 6). In some embodiments, the mosaic crRNA is 80%, 85%, 90%, or 95% identical to TatH-GCUUAGGCAUCUCCUAUGGC (SEQ ID NO: 7). In some embodiments, the mosaic crRNA is 80%, 85%, 90%, or 95% identical to TatI-GGCUCUAGGUUAGGAUCUAC (SEQ ID NO: 8).

In some embodiments, a mosaic crRNA disclosed herein reduces HIV-1 replication by at least 50%. In some embodiments, a mosaic crRNA disclosed herein reduces HIV-1 replication by at least 60%, 65%, 70%, 75%, 80%, 85%, 90%, 95%, or 99%. In some embodiments, TatD reduces HIV-1 replication by at least 54%. In some embodiments, TatE reduces HIV-1 replication by 76%. In some embodiments, co-administration of TatD and TatE (TatDE) reduces HIV-1 replication by an average of 82% in 7 strains, including 6 clade B transmitted founder strains.

In some embodiments, a mosaic crRNA disclosed herein is effective against at least 50% of HIV-1 strains. In some embodiments, a mosaic crRNA disclosed herein is effective against at least 60%, 65%, 70%, 75%, 80%, 85%, 90%, 95%, or 99% of HIV-1 strains. In some embodiments, TatDE therapy is effective against at least 62% of all HIV-1 strains.

In some embodiments, a crRNA disclosed herein is operable with any suitable Cas enzyme. In some embodiments, a crRNA disclosed herein is operable with a Cas enzyme selected from the group consisting of: Cas9, CasPhi (CasΦ), Cas3, Cas8a, Cas5, Cas8b,

Cas8c, Cas10d, Cse1, Cse2, Csy1 Csy2, Csy3, Cas10, Csm2, Cmr5, Cas10, Csx11, Csx10, Csf1. Csn2, Cas4, C2c1, C2c3, Cas12a (Cpf1), Cas12b, Cas12e, Cas13a, Cas13, Cas13c, and Cas13d. In some embodiments, a crRNA disclosed herein is operable with Cas9.

In some embodiments, a crRNA disclosed herein is part of a single guide RNA (“sgRNA”) sequence wherein the sgRNA sequence comprises the crRNA sequences and a tracrRNA sequence. Any suitable tracrRNA sequence is contemplated for use with a sgRNA disclosed herein. In some embodiments, the sgRNA comprises TatA2 and a tracrRNA sequence. In some embodiments, the sgRNA comprises TatD and a tracrRNA sequence. In some embodiments, the sgRNA comprises TatE and a tracrRNA sequence. In some embodiments, the sgRNA comprises TatE2 and a tracrRNA sequence. In some embodiments, the sgRNA comprises TatF and a tracrRNA sequence. In some embodiments, the sgRNA comprises TatG and a tracrRNA sequence. In some embodiments, the sgRNA comprises TatH and a tracrRNA sequence. In some embodiments, the sgRNA comprises TatI and a tracrRNA sequence.

In some embodiments, the crRNA sequence is a DNA sequence (such as single-or double stranded linear sequences; or plasmid DNA), an RNA sequence, or a recombinantly expressed crRNA/protein fusion (such as ribonucleoprotein (RNP)). In some embodiments, the DNA or RNA sequence comprising the crRNA sequence further comprises a tracrRNA sequence (e.g., a sgRNA sequence) and/or a sequence encoding a Cas9 enzyme.

CRISPR-Cas9 based therapeutics include but are not limited to CRISPR-Cas9 ribonucleoprotein (RNPs), guide RNAs and/or crRNAs that target or are complementary to a sequence within one or more HIV-1 genes including but not limited to tat, rev, env-gp41, gag-p1, gag-p6, vif, vpr, vpu, and nef, and plasmids or other constructs containing the guide RNAs and/or crRNAs. In some embodiments, the guide RNAs and/or crRNAs include but are not limited to TatD, TatH, TatE, TatE2, TatA2, TatG, TatF, and/or combinations thereof, and/or plasmids containing TatD, TatH, TatE, TatE2, TatA2, TatG, TatF and/or combinations thereof or RNPs containing TatD, TatH, TatE, TatE2, TatA2, TatG, TatF, and/or combinations thereof as described in PCT/US2021/021246 (incorporated by reference herein) and/or mRNAs containing TatD, TatH, TatE, TatE2, TatA2, TatG, TatF and/or combinations thereof. In some embodiments the therapeutic agent is a combination of TatD and TatE guide RNAs and/or crRNAs, plasmids containing TatD and TatE guide RNAs or crRNAs, and/or RNPs containing TatD and TatE guide RNAs and/or crRNAs (the combination of TatD and TatE may be referred to as TatDE), or mRNAs containing TatD and TatE guide RNAs and/or crRNAs. In some embodiments, the CRISPR-Cas9 base therapeutic is encapsulated by the cationic lipid, which is in turn encapsulated by the remaining lipids (such as the zwitterionic lipid, the PEG-lipid conjugates, and the sterol). In some embodiments, the crRNA loaded into the lipid nanoparticle is selected from SEQ ID NO: 1-8. In some embodiments the crRNA loaded into the lipid nanoparticle is crRNA encoding for TatDE. In some embodiments the crRNA loaded into the lipid nanoparticle is selected from SEQ ID NO: 2 and SEQ ID NO: 3. In some embodiments, the crRNA sequence is encoded in a vector. In some embodiments the crRNA is a mRNA.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is formulated as a lipid nanoparticle (LNP). A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

In some embodiments, the composition comprises: TatD and TatH (TatD/H). In some embodiments, the composition comprises: TatD and TatE (TatD/E). In some embodiments, the composition comprises: TatE and TatH (TatE/H). In some embodiments, the composition comprises: TatD and TatA2 (TatA2/D).

In some embodiments, the lipid nanoparticle composition comprises: TatD/tracrRNA and TatH/tracrRNA. In some embodiments, the lipid nanoparticle composition comprises: TatD/tracrRNA and TatE/tracrRNA. In some embodiments, the lipid nanoparticle composition comprises: TatE/tracrRNA and TatH/tracrRNA. In some embodiments, the lipid nanoparticle composition comprises: TatD/tracrRNA and TatA2/tracrRNA.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is part of a vector. In some embodiments, the Cas enzyme is part of a vector. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA. TatE2/tracrRNA, TatF/tracrRNA, TatG/tracrRNA, TatH/tracrRNA, or TatI/tracrRNA) and the Cas enzyme are part of the same vector. In some embodiments, the crRNA disclosed here (any of TatA2, TatD, TatE, TatE2, TatF, TatG, TatH, or TatI) or sgRNA disclosed herein (any of TatA2/tracrRNA. TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of separate vectors.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is part of a mRNA. In some embodiments, the Cas enzyme is part of a mRNA. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of the same mRNA. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are part of separate mRNAs.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is enveloped in a LNP. In some embodiments, the Cas enzyme enveloped in a LNP. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are enveloped in the same LNP. In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are enveloped in separate LNPs.

In some embodiments, a crRNA disclosed here or sgRNA disclosed herein is part of a vector. In some embodiments, the Cas enzyme is part of a vector. In some embodiments, the crRNA disclosed here or sgRNA disclosed herein and the Cas enzyme are part of the same vector. In some embodiments, the crRNA disclosed here or sgRNA disclosed herein and the Cas enzyme are part of separate vectors.

In some embodiments, a crRNA or sgRNA disclosed here is part of a mRNA. In some embodiments, the Cas enzyme is part of a mRNA. In some embodiments, the crRNA disclosed here or sgRNA disclosed herein and the Cas enzyme are part of the same mRNA. In some embodiments, the crRNA disclosed here or sgRNA disclosed herein and the Cas enzyme are part of separate mRNAs.

In some embodiments, a crRNA disclosed here or sgRNA disclosed herein is enveloped in a LNP. In some embodiments, the Cas enzyme enveloped in a LNP. In some embodiments, the crRNA disclosed here or sgRNA disclosed herein and the Cas enzyme are enveloped in the same LNP. In some embodiments, the crRNA disclosed here (any of TatA2, TatD, TatE, TatE2, TatF, TatG, TatH, or TatI) or sgRNA disclosed herein and the Cas enzyme are enveloped in separate LNPs.

### Pharmaceutical Compositions

Disclosed herein, in some embodiments are pharmaceutical compositions comprising: (a) lipid nanoparticles comprising a plurality of lipids, a CXCR4 targeting moiety, and a CRISPR nucleic acid complementary to a sequence within an HIV-1 gene, and (b) a pharmaceutically acceptable excipient.

In some embodiments, the pharmaceutical composition comprises (a) a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA), and (b) a pharmaceutically acceptable excipient. In some embodiments, the composition further comprises a Cas enzyme.

In some embodiments. the pharmaceutical composition comprises: TatD and TatH (TatD/H). In some embodiments, the pharmaceutical composition comprises: TatD and TatE (TatD/E). In some embodiments, the pharmaceutical composition comprises: TatE and TatH (TatE/H). In some embodiments, the pharmaceutical composition comprises: TatD and TatA2 (TatA2/D).

In some embodiments, the pharmaceutical composition comprises: TatD/tracrRNA and TatH/tracrRNA. In some embodiments, the pharmaceutical composition comprises: TatD/tracrRNA and TatE/tracrRNA. In some embodiments, the pharmaceutical composition comprises: TatE/tracrRNA and TatH/tracrRNA. In some embodiments, the pharmaceutical composition comprises: TatD/tracrRNA and TatA2/tracrRNA.

In some embodiments, a crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) is a part of a vector (such as a viral vector). In some embodiments, the Cas enzyme is a part of a vector (such as a viral vector). In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are parts of the same vector (such as a viral vector). In some embodiments, the crRNA disclosed here (any of TatA2, TatD, TatE, TatE2, TatF, TatG, TatH, or TatI) or sgRNA disclosed herein (any of TatA2/tracrRNA, TatD/tracrRNA, TatE/tracrRNA, TatE2/tracrRNA, TatF/tracrRNA, TatG/tracrRNA, TatH/tracrRNA, or TatI/tracrRNA) and the Cas enzyme are parts of separate vectors (such as viral vectors).

In some embodiments, the pharmaceutically acceptable excipient is a carrier, solvent, stabilizer, adjuvant, diluent, etc., depending upon the particular mode of administration and dosage form.

Suitable excipients include, for example, carrier molecules that include large, slowly metabolized macromolecules such as proteins. polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, cellulose, dextrin).

In some embodiments, the composition has a physiologically compatible pH (e.g., a range from a pH of about 3 to a pH of about 11, about pH 3 to about pH 7, depending on the formulation and route of administration). In some cases, the pH is from about pH 5.0 to about pH 8.

In some embodiments, the composition further comprises a second active ingredient useful in the treatment or prevention of bacterial growth (for example and without limitation, anti-bacterial or anti-microbial agents).

### Methods of Use

Disclosed herein, in some embodiments, are methods of treating an HIV-1 infection in an individual in need thereof. Further disclosed herein, in some embodiments, are methods of preventing an HIV-1 infection in an individual in need thereof. Additionally, disclosed herein, in some embodiments, are methods of preventing transmission of an HIV-1 virus from one individual to another (for example, from a pregnant woman to a child, for example during birth or breast feeding).

Also provided herein, in some embodiments, are methods of disrupting the transcription of an exon of an HIV-1 sequence in an individual in need thereof.

Further disclosed herein, in some embodiments, are methods of excising all or a portion of an HIV genome in an HIV infected cell of an individual.

In some embodiments, the methods comprise administering to the individual a lipid nanoparticle comprising a plurality of lipids, a CXCR4 targeting moiety, and a CRISPR nucleic acid complementary to a sequence within an HIV-1 gene, or a pharmaceutical composition comprising said lipid nanoparticles and a pharmaceutically acceptable carrier, as disclosed herein. In some embodiments, the crRNA comprises any of TatA2, TatD, TatE, TatE2, TatF, TatG, TatH, or TatI. In some embodiments, the methods comprise administering to an individual any lipid nanoparticle comprising a combination of a crRNA disclosed here. In some embodiments, the method comprises administering to the individual: TatD and TatH (TatD/H). In some embodiments, the method comprises administering to the individual: TatD and TatE (TatD/E). In some embodiments, the method comprises administering to the individual: TatE and TatH (TatE/H). In some embodiments, the method comprises administering to the individual: TatD and TatA2 (TatA2/D).

In some embodiments, the methods comprise administering to an individual any sgRNA disclosed herein (any of TatA2/tracrRNA, TatD/tracrRNA, TatE/tracrRNA, TatE2/tracrRNA, TatF/tracrRNA, TatG/tracrRNA. TatH/tracrRNA, or TatI/tracrRNA). In some embodiments, the methods comprise administering to an individual any combination of a sgRNA disclosed here. In some embodiments, the method comprises administering to the individual: TatD/tracrRNA and TatH/tracrRNA. In some embodiments, the method comprises administering to the individual: TatD/tracrRNA and TatE/tracrRNA. In some embodiments, the method comprises administering to the individual: TatE/tracrRNA and TatH/tracrRNA. In some embodiments, the method comprises administering to the individual: TatD/tracrRNA and TatA2/tracrRNA.

In some embodiments, the methods dysregulate virion production from a latent proviral DNA or impede integration of reverse-transcribed proviral DNA. In some embodiments, the crRNA is a mosaic crRNA. In some embodiments, the crRNA binds to a plurality of nucleic acids within an overlapping exon of at least two HIV-1 genes. In some embodiments, the crRNA binds to a plurality of nucleic acids within an overlapping exon of at least three HIV-1 genes.

A pharmaceutical composition disclosed herein is administered by any appropriate route that results in effective treatment in the subject. In some embodiments, a pharmaceutical composition disclosed herein is administered systemically. In some embodiments, a pharmaceutical composition disclosed herein is administered locally. The pharmaceutical composition is administered via a route such as, but not limited to, enteral, gastroenteral, oral, transdermal, subcutaneous, nasal, intravenous, intravenous bolus, intravenous drip, intraarterial, intramuscular, transmucosal, insufflation, sublingual, buccal, conjunctival, cutaneous. Modes of administration include injection, infusion, instillation, and/or ingestion. “Injection” includes, without limitation, intravenous, intramuscular, intra-arterial, intrathecal, intraventricular, intradermal, intraperitoneal, transtracheal, and subcutaneous. In some examples, the route is intravenous.

### EXAMPLES

### Example 1. Synthesis of DSPE-PEG-AMD070 Conjugate and Lipid Nanoparticles

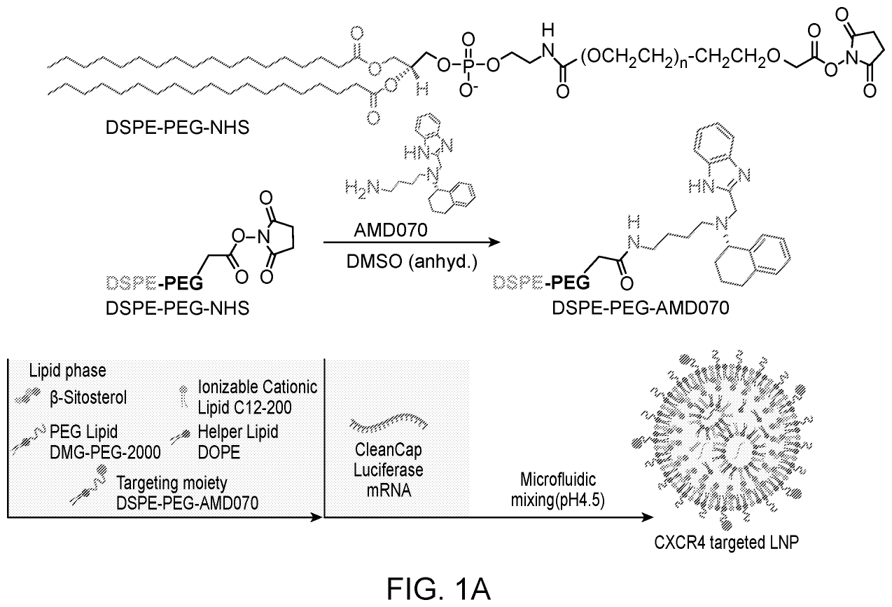

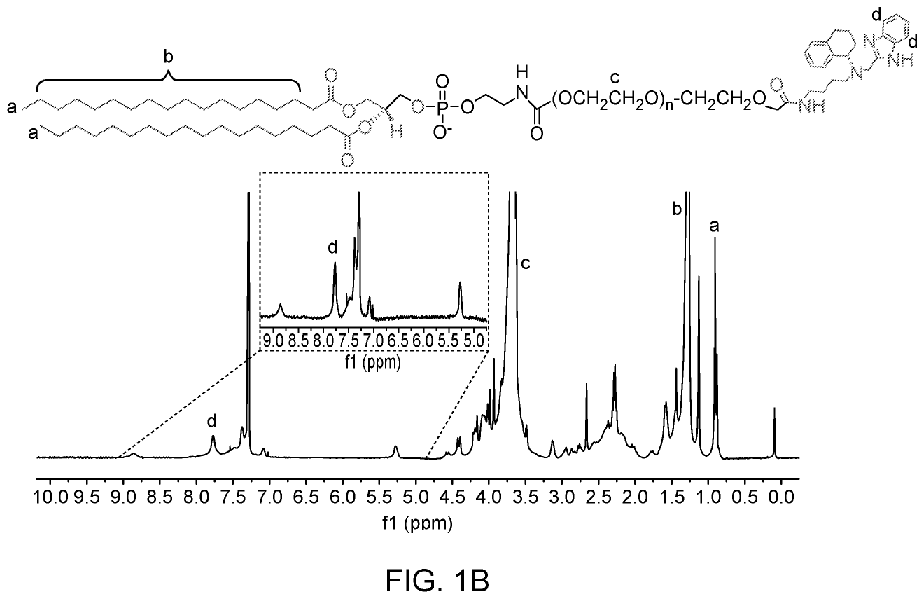

FIG. 1A illustrates an exemplary synthetic scheme of a CXCR4 targeting conjugate and lipid nanoparticles comprising said CXCR4 targeting conjugate. AMD070, a CXCR4 targeting small molecule, was conjugated with DSPE-PEG through an acid-amine coupling reaction. FIG. 1B is an ¹H NMR spectrum (in CDC13) of the DSPE-PEG-AMD070 conjugate. In FIG. 1B, the NMR signals corresponding to DSPE, PEG and AMD070 protons are designated by a and b, c, and d, respectively.

The DSPE-PEG-AMD070 conjugate was integrated with ionizable cationic lipids (C12-200), zwitterionic lipids (DOPE), PEG lipids (DMG-PEG), β-sitosterol, and CleanCap® luciferase mRNA (luc mRNA), and formulated into targeted ligands on LNPs (referred to as T-LNPs). The T-LNPs showed an average particle size (83 nm) larger than the average particle size (69 nm) of LNP without AMD070 (the lipid nanoparticles without a CXCR4 targeting small molecular ligand are referred to as C-LNP), thus illustrating successful integration of AMD070 in T-LNPs. The T-LNPs demonstrated an mRNA encapsulation efficiency (EE) of 79% which was comparable with EE of C-LNP (74%).

### Example 2. Targeted Delivery of RNA to CXCR4 expressing CD4+ T cells by DSPE-PEG-AMD070 Lipid Nanoparticles

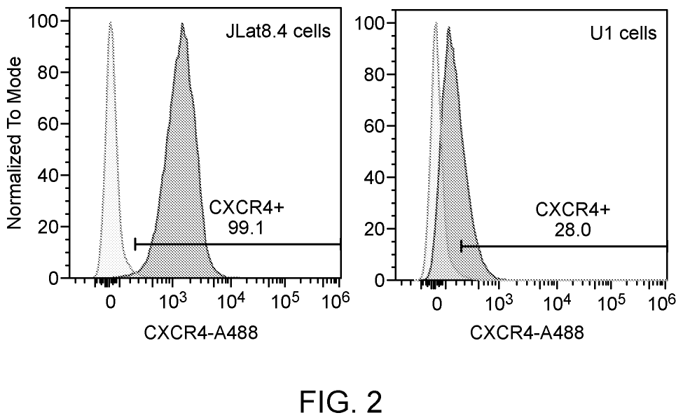

To demonstrate CXCR4 targeted delivery, a high CXCR4 expressing (˜99% CXCR4 expressing cell, FIG. 2) that harbor HIV-1 proviral DNA in a latent state was used. This CD4+ T cell J-Lat8.4 cell line and low level CXCR4 myeloid expressor (˜28% CXCR4 positive cell, FIG. 2) were studied in these assays. The myeloid cell line was named U1. It is a pro-monocytic cell that also harbors integrated proviral HIV DNA. Both the J-Lat8.4 and U1 cells were readily propagated (FIG. 3) with 80% vitality when administered with dosages of 0.16 and 0.8 μg/million cells of luc mRNA. respectively. Moreover, LNP-mediated luc expression on J-Lat8.4 and U1 cells demonstrated that T-LNPs and C-LNPs showed a dose-dependent increase in luc expression (FIG. 4). A high-dose toxicity-induced decrease in luc expression in J-Lat8.4 and U1 cells was also demonstrated.

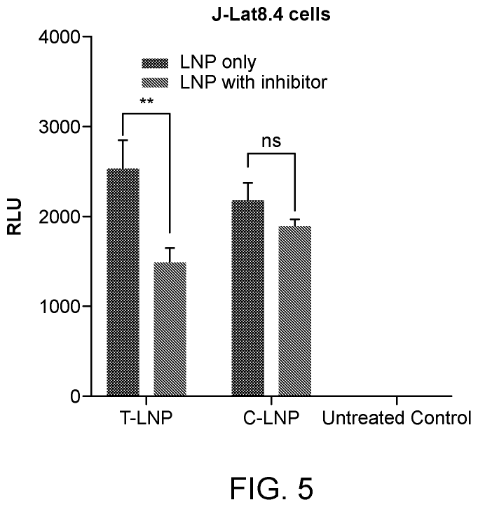

The results demonstrate that T-LNPs deliver luc mRNA to CXCR4 expressing CD4+ T cells. In comparison with C-LNPs, T-LNPs showed greater than 2-fold increase in luc expression (FIG. 4) selectively in high CXCR4 expressing J-Lat8.4 cells at luc mRNA dosage of 0.16 μg/million cells. In marginally CXCR4 expressing U1 cells, T-LNPs showed a similar level of luc expression (FIG. 4) as with C-LNP. These results demonstrate that T-LNPs are more efficient and selective in delivering luc mRNA to the CXCR4 expressing J-Lat8.4 cells. Moreover, T-LNPs treated J-Lat8.4 cells showed a significant decrease in luciferase expression (FIG. 5) after blocking the receptor by preincubation with the CXCR4 inhibitor. These results further support the CXCR4 receptor mediated mRNA delivery of T-LNPs to J-Lat8.4 cells.

### Example 3. Targeted Delivery of RNA to CXCR4 Expressing CD4+ T Cells by DSPE-PEG-CycPep Lipid Nanoparticles

Clustered regularly interspaced short palindromic repeats (CRISPR) guide RNAs (gRNAs) which target HIV-1gp41, tat, and rev exons are exemplified herein. T-LNPs were formulated that carried luc mRNA with an encapsulation efficiency greater than 97%. Cell targeting was seen based on Luc expression. CRISPR-Cas9/HIV-1gRNA loaded T-LNPs demonstrated viral excision of up to 60% commensurate with CXCR4 expression levels in J-Lat 8.4 and JE6 (CD4+ T cells) and U1 (monocytic) cells. LNPs with a helper which produced a tropic shift from liver to the spleen and lymph nodes were formulated. The ability to target HIV-1 reservoirs was demonstrated by T-LNP loaded with Luc mRNA in mice.

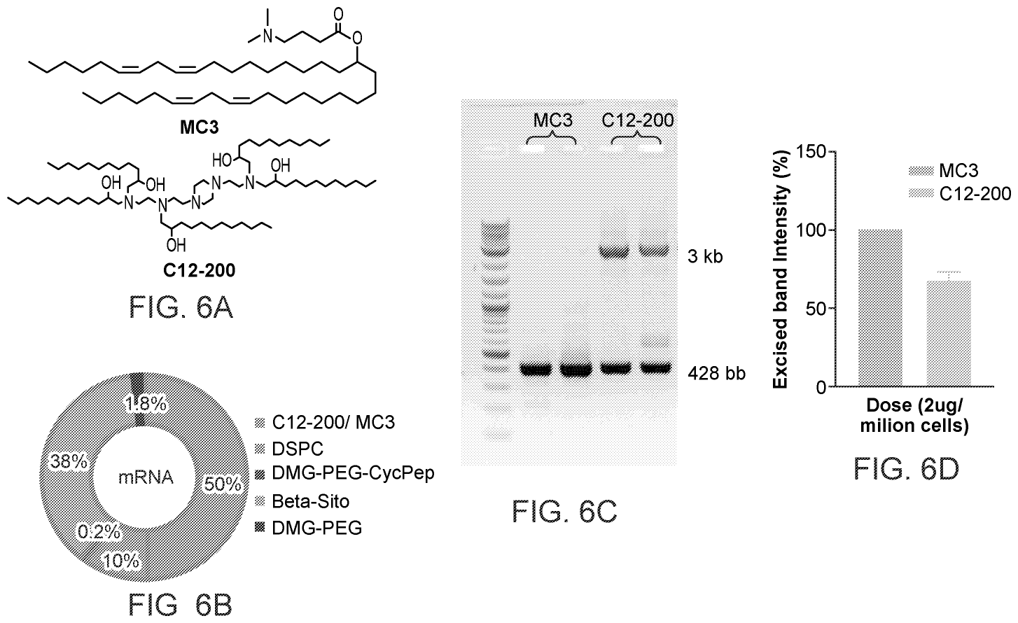

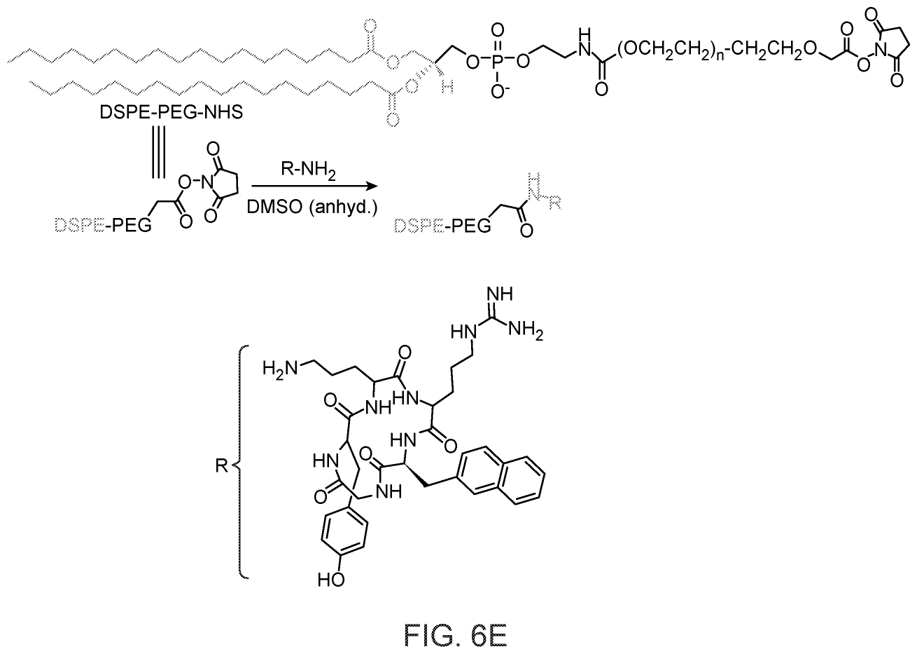

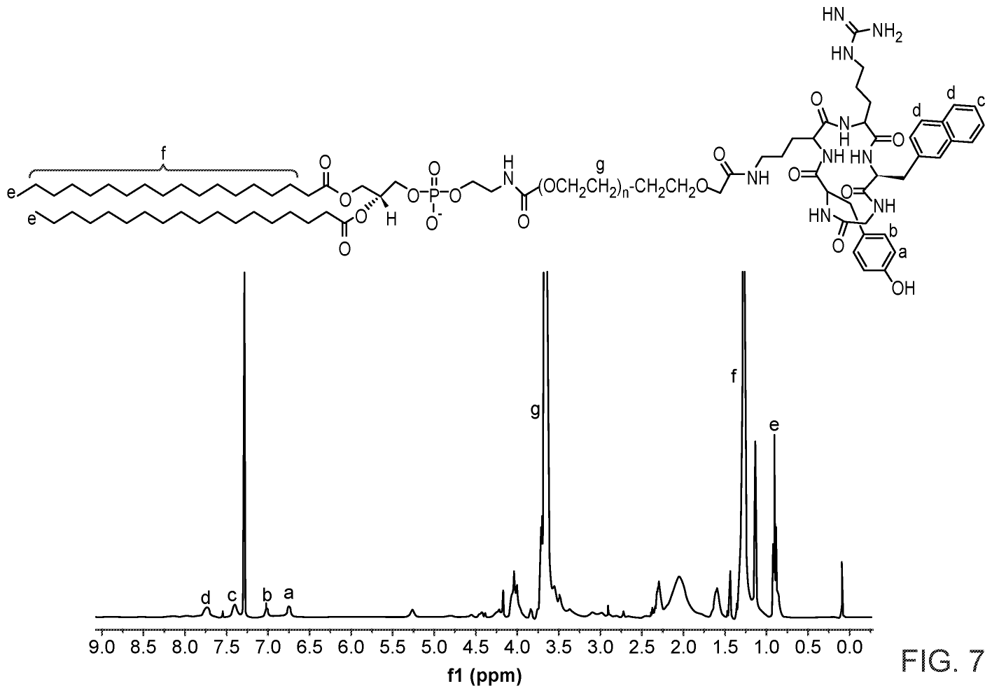

For example, LNPs were formulated using the combination of helper lipid (DSPC), PEG lipids (DMG-PEG) and β-sitosterol with CRISPR-Cas9/gRNA and varying the ionizable lipid (LP01 or 3060110 or DLin-KC2-DMA or C12-200 or MC3). LNPs with MC3 exhibited the highest viral DNA excision efficacy in the latently infected CD4+ T cells (J-Lat8.4). FIG. 6A shows chemical structures of MC3 and C12-200 ionizable lipids. FIG. 6B shows a pie chart representing the lipid components used to formulate lipid nanoparticles. DNA was extracted from JLat 8.4 cell line 72 hours after treatment with MC3 and C12-200 containing LNPs at a dose of 2 μg/million cells. The gel image of FIG. 6C displays the intact proviral DNA band (3 kb) and the excised band (428 bp). The excised band intensity on the gel blot of FIG. 6D was quantified using ImageJ software and plotted using GraphPad Prism 10.0.0. A cyclic peptide with the amino acid sequence cyclo (D-Tyr-Orn-Arg-Nal-Gly-) (CycPep) as a potential CXCR4 targeting ligand was synthesized and functionalized with DSPE-PEG by adopting acid-amine coupling reaction. FIG. 6E is a schematic presentation of the synthesis of DSPE-PEG-CycPep by activated acid-amine coupling reaction. The chemical structure of the DSPE-PEG-CycPep was confirmed by ¹H NMR spectrum is shown in FIG. 7.

The DSPE-PEG-CycPep was integrated with the final LNP composition (MC3, DSPC, DMG-PEG, and β-sitosterol) to accomplish luc mRNA encapsulated, CXCR4 targeted LNP (T-LNP). LNP without DSPE-PEG-CycPep (C-LNP) was also formulated and used as a control for various studies.

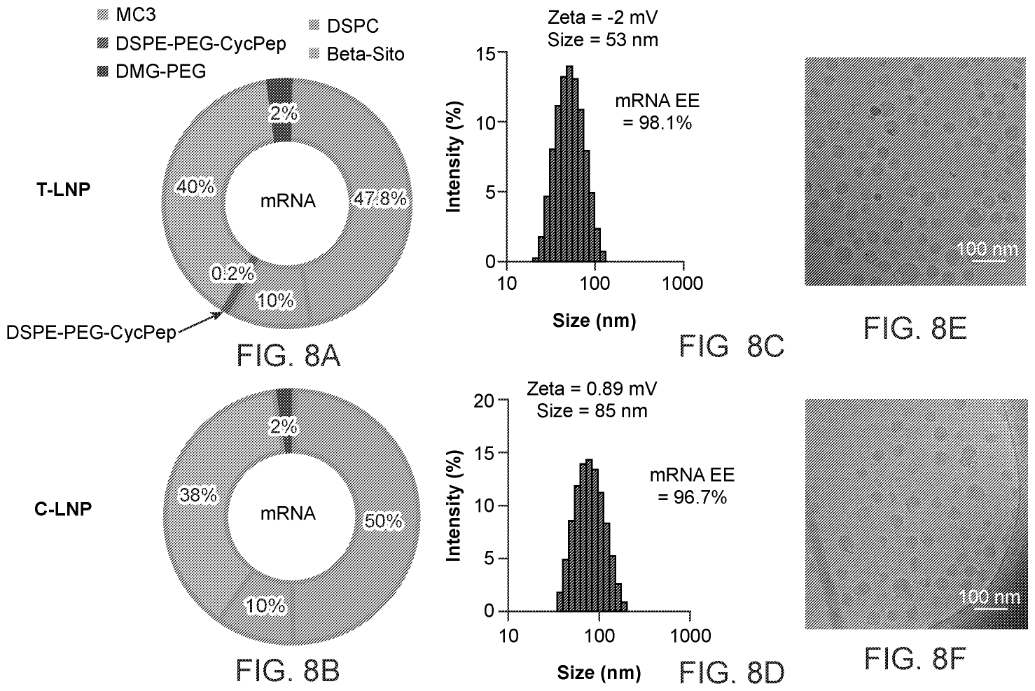

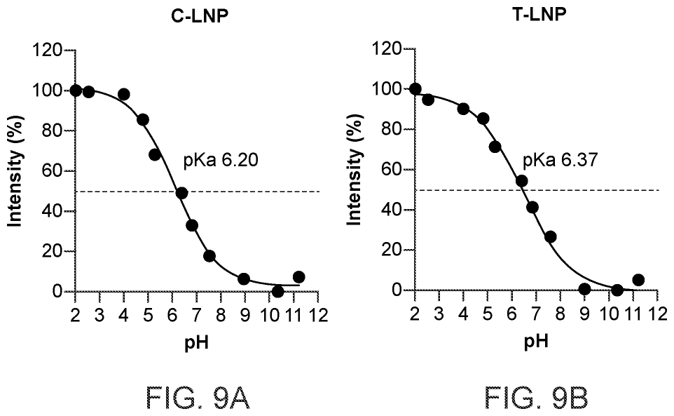

The DSPE-PEG-CycPep was integrated with the final LNP composition (MC3, DSPC, DMG-PEG, and-sitosterol) to accomplish luc mRNA encapsulated, CXCR4 targeted LNP (T-LNP). LNP without DSPE-PEG-CycPep (C-LNP) was also formulated and used as a control for various studies (FIG. 8A, FIG. 8B). Both the T-LNP and C-LNP have size below: 100 nm with narrow polydispersity index and nearly neutral zeta potential, suitable for intravenous mRNA delivery (FIG. 8C, FIG. 8D). Cryo-EM showed a spherical morphology of LNPs (FIG. 8E, FIG. 8F). Both the LNPs showed pka between 6.20 to 6.40 (analyzed by TNS assay) suitable for endosomal escape and cytosolic delivery of mRNA (FIG. 9A, FIG. 9B). The cell viability of LNPs was evaluated against J-Lat8.4, Jurkat E6 (express 99% CXCR4) and U1 (express 28% CXCR4) cell lines.

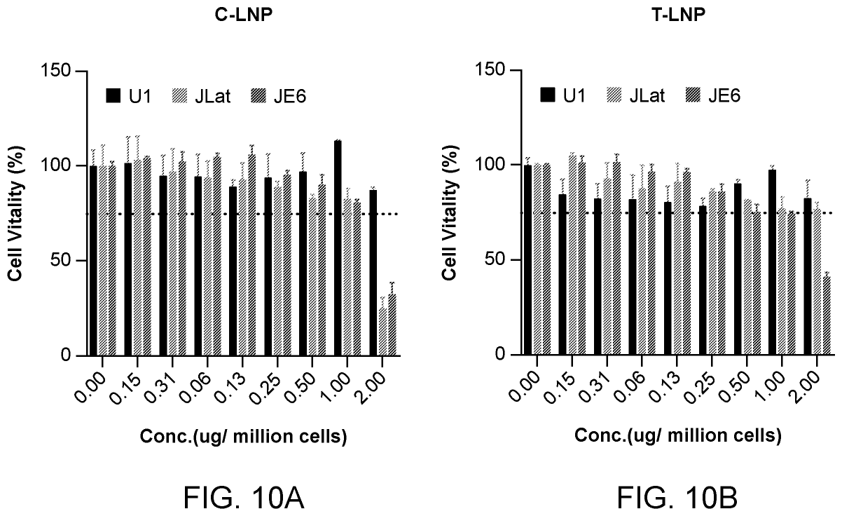

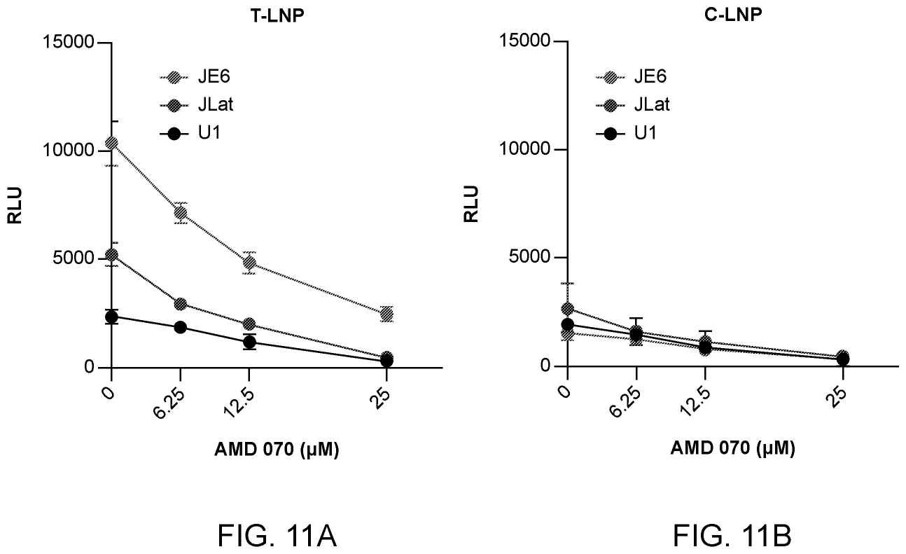

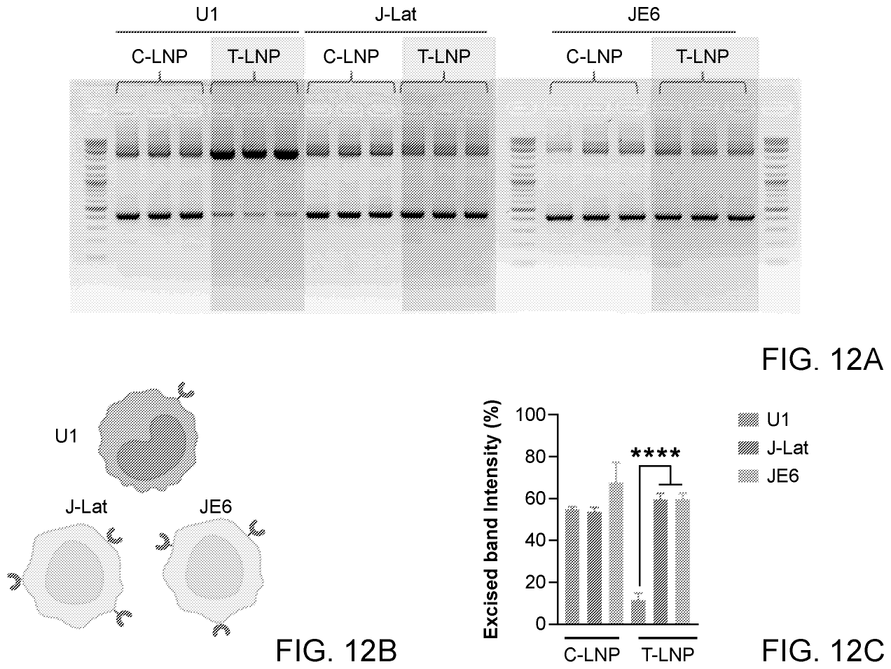

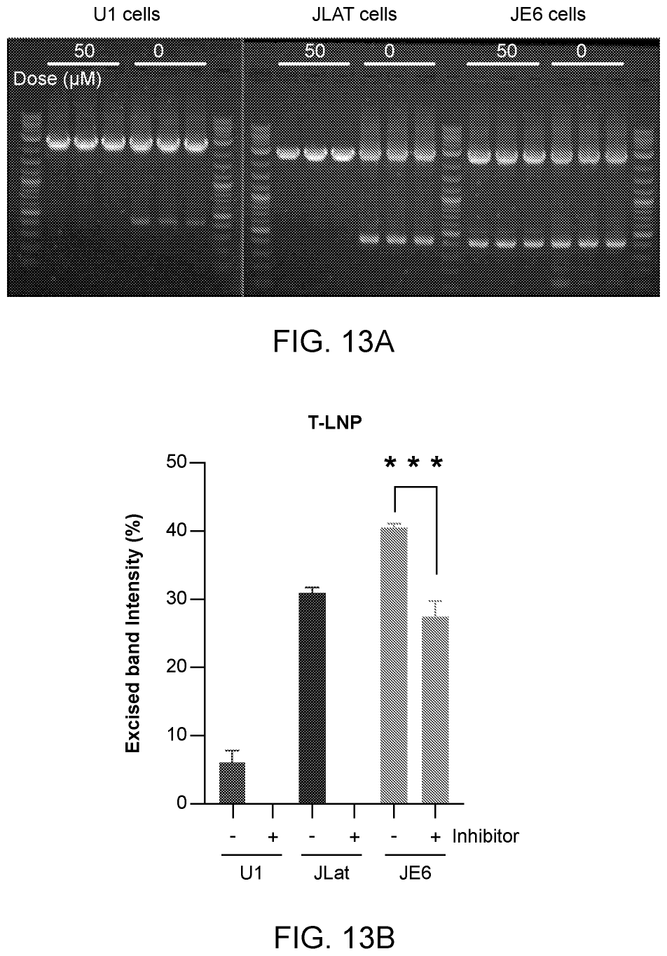

LNPs showed cell viability above 70% at the mRNA dose of 1 μg/million cells which has been considered the maximum safe dose to evaluate the therapeutic performance (FIG. 10A, FIG. 10B). In comparison with C-LNP, T-LNP treated cells demonstrated a higher level of FLuc expression throughout all the cell lines, indicating its profound mRNA-delivering proficiency. Furthermore, to determine whether this delivery was CXCR4-specific or not, cells were preincubated with AMD070 (CXCR4 blocker) followed by the treatment with LNPs. Unlike C-LNP, T-LNP showed a substantial decrease in FLuc expression with increasing AMD070 concentration (FIG. 11A, FIG. 11B). These results showed that CXCR4-expressing cell-specific mRNA delivery of T-LNP. Furthermore, CRISPR-Cas9/gRNA encapsulated LNPs were prepared. The viral DNA excision efficiency was evaluated by treating both LNPs on U1, J-Lat8.4, and Jurkat E6 cell lines at the aforementioned dose. The C-LNP showed nonspecific excision of viral DNA as it demonstrated a similar level of excision efficiency throughout all types of cell lines irrespective of their level of CXCR4 expression (FIG. 12A, FIG. 12B, FIG. 12C). However, T-LNP showed a cell-specific DNA excision as it demonstrated a 4-fold higher excision in highly CXCR4 expressing J-Lat8.4 and Jurkat E6 cell lines, in comparison with moderately CXCR4 expressing U1 cell line. Further to confirm the CXCR4-specific DNA excision, cells were preincubated (for 30 min.) with AMD070 and treated with T-LNP. The result revealed a substantial decrease in DNA excision in the presence of AMD070 (FIG. 13A, FIG. 13B). This result further confirmed the CXCR4-specific DNA excision of T-LNP in differentially CXCR4-expressed cell lines.

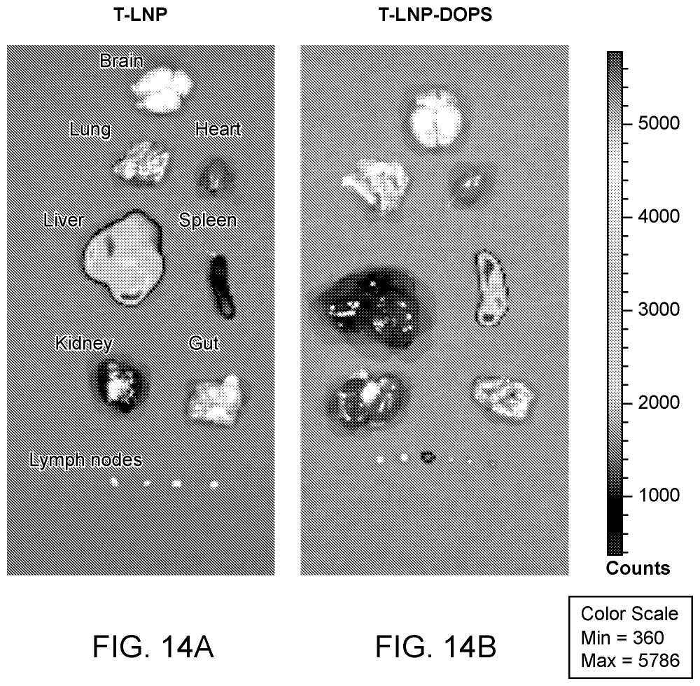

DOPS (spleen targeting helper lipid) with our existing T-LNP composition, formulated a compositionally unique T-LNP-DOPS loaded with luc mRNA, intravenously injected into the 8 weeks old BALB/cJ mice, and observed the protein expression under IVIS. Unlike T-LNP, T-LNP-DOPS shows FLuc expression only in the spleen and lymph node, the secondary lymphoid organs served as HIV-1 reservoirs (FIG. 14A, FIG. 14B). The HIV-1 reservoir-specific mRNA delivery system is translated for Cas9/gRNA delivery system to eliminate the viral DNA from that HIV-1 reservoir.

1. A lipid nanoparticle, comprising:
(a) a shell comprising a plurality of lipids and having
(i) an exterior surface comprising a CXCR4 targeting moiety linked to a PEG-lipid conjugate; and
(ii) an interior surface defining an inner cavity; and
(b) a CRISPR nucleic acid complementary to a sequence within an HIV-1 gene encapsulated within the inner cavity of the shell.

2. The lipid nanoparticle of claim 1, wherein the plurality of lipids comprises cationic lipids, zwitterionic lipids, sterol, and PEG-lipid conjugates.

3. The lipid nanoparticle of claim 1 or 2, further comprising a spleen targeting helper lipid.

4. The lipid nanoparticle of any one of claims 1-3, wherein the lipid nanoparticle comprises C12-200, DOPE, β-sitosterol, and DMG-PEG.

5. The lipid nanoparticles of any one of claims 1-3, wherein the lipid nanoparticle comprises MC3, DSPC, DMG-PEG, β-sitosterol, and DOPS.

6. The lipid nanoparticle of claim 1, wherein the CXCR4 targeting moiety is a CXCR4 inhibitor.

7. The lipid nanoparticle of claim 1, wherein the CXCR4 targeting moiety is AMD3100, AMD3465, IT1t, KRH-3955, AMD070, HF51116, BPRCX807, or cyclo-[Nal-Gly-(D-Tyr)-Or-Arg], or pharmaceutically acceptable salts thereof.

8. The lipid nanoparticle of claim 7, wherein the CXCR4 targeting moiety is AMD070, or a pharmaceutically acceptable salt thereof.

9. The lipid nanoparticle of claim 7, wherein the CXCR4 targeting moiety is cyclo-[Nal-Gly-(D-Tyr)-Or-Arg], or a pharmaceutically acceptable salt thereof.

10. The lipid nanoparticle of any one of claims 1-9, wherein the PEG-lipid conjugate is DSPE-PEG.

11. The lipid nanoparticle of any one of claims 1-10, comprising a crRNA sequence that is complementary to a plurality of nucleic acids of a consensus sequence of an HIV-1 gene selected from the group consisting of: tat, rev, env-gp41, gag-p1, gag-p6, vif, vpr, vpu, and nef.

12. The lipid nanoparticle of claim 11, wherein the crRNA sequence is adjacent to a PAM sequence.

13. The lipid nanoparticle of claim 11, wherein the crRNA sequence is complementary to a plurality of nucleic acids of an overlapping sequence.

14. The lipid nanoparticle of any one of claims 1-13, wherein the nucleic acid sequence comprises two crRNA sequences, each sequence complementary to a plurality of nucleic acids of a consensus sequence of an HIV-1 gene selected from the group consisting of: tat, rev, env-gp41, gag-p1, gag-p6, vif, vpr, vpu, and nef; wherein the crRNA sequences are not complementary to the same sequences.

15. The lipid nanoparticle of claim 14, wherein the overlapping sequence is part of a nucleic acid sequence of at least two HIV-1 genes selected from the group consisting of: tat, rev, env-gp41, gag-p1, gag-p6, vif, vpr, vpu, and nef.

16. The lipid nanoparticle of claim 14, wherein the overlapping sequence is part of a nucleic acid sequence of at least three HIV-1 genes selected from the group consisting of: tat, rev, env-gp41, gag-p1, gag-p6, vif, vpr, vpu, and nef.

17. The lipid nanoparticle of claim 14, wherein the overlapping exon is part of a nucleic acid sequence selected from the group consisting of tat (exon 1, nucleic acids 5831-6045; exon 2, nucleic acids 8379-8469), rev (exon 1, nucleic acids 5970-6045; or exon 2, nucleic acids 8379-8653), env-gp41 (nucleic acids 7758-8795), gag-p1 (nucleic acids 2086-2134), gag-p6 (nucleic acids 2134-2292), vif (nucleic acids 5041-5619), vpr (nucleic acids 5559-5850), vpu (nucleic acids 6045-6310), and nef (nucleic acids 8797-9417).

18. The lipid nanoparticle of claim 14, wherein the overlapping sequence is nucleic acids 7758-8795 of HIV-1 gene gp41-env, exon 2 (nucleic acids 8379-8469) of HIV-1 gene tat, and exon 2 (nucleic acids 8379-8653) of HIV-1 gene rev.

19. The lipid nanoparticle of claim 14, wherein the overlapping exon is exon 1 (nucleic acids 5831-6045) of HIV-1 gene tat, and exon 1 (nucleic acids 5970-6045) of HIV-1 gene rev.

20. The lipid nanoparticle of claim 11, wherein the crRNA has a sequence at least 80% identical to SEQ ID NO: 1.

21. The lipid nanoparticle of claim 11, wherein the crRNA has a sequence at least 80% identical to SEQ ID NO: 2.

22. The lipid nanoparticle of claim 11, wherein the crRNA has a sequence at least 80% identical to SEQ ID NO: 3.

23. The lipid nanoparticle of claim 11, wherein the crRNA has a sequence at least 80% identical to SEQ ID NO: 4.

24. The lipid nanoparticle of claim 11, wherein the crRNA has a sequence at least 80% identical to SEQ ID NO: 5.

25. The lipid nanoparticle of claim 11, wherein the crRNA has a sequence at least 80% identical to SEQ ID NO: 6.

26. The lipid nanoparticle of claim 11, wherein the crRNA has a sequence at least 80% identical to o SEQ ID NO: 7.

27. The lipid nanoparticle of claim 11. wherein the crRNA has a sequence at least 80% identical to SEQ ID NO: 8.

28. The lipid nanoparticle of claim 11, wherein the nucleic acid encodes for a TatDE crRNA.

29. The lipid nanoparticle of claim 28, wherein the TatDE crRNAs comprise SEQ ID NO: 2 and SEQ ID NO: 3.

30. The lipid nanoparticle of any one of claims 1-29, wherein the nucleic acid sequence further comprises a tracrRNA sequence.

31. The lipid nanoparticle of any one of claims 1-30, wherein the nucleic acid sequence further comprises a sequence that encodes a Cas protein.

32. The lipid nanoparticle of claim 31, wherein the nucleic acid encoding for a Cas protein is a part of a vector and the nucleic acid encoding for the crRNA is a part of a vector.

33. The lipid nanoparticle of claim 31. wherein the nucleic acid encoding for a Cas protein is a mRNA and the nucleic acid encoding for the crRNA is a mRNA.

34. The lipid nanoparticle of claim 31, wherein the Cas protein is a Cas9, CasPhi (Cas Φ), Cas3, Cas8a, Cas5, Cas8b, Cas8c, Cas10d, Cse1, Cse2, Csy1 Csy2, Csy3, Cas10, Csm2, Cmr5, Cas10, Csx11, Csx10, Csf1, Csn2, Cas4, C2c1, C2c3, Cas 12a (Cpf1), Cas12b, Cas12e, Cas13a, Cas13, Cas13c, or Cas13d.

35. The lipid nanoparticle of claim 34, wherein the Cas protein is a Cas9 protein.

36. The lipid nanoparticle of claim 35, where the nucleic acid encoding for Cas9 is a part of a vector and the nucleic acid encoding for TatDE crRNAs is a part of a vector.

37. The lipid nanoparticle of claim 35, where the nucleic acid encoding for Cas9 is a mRNA and the nucleic acid encoding for TatDE crRNAs is a mRNA.

38. The lipid nanoparticle of any one of claims 1-37, wherein the nucleic acid sequence is a DNA sequence.

39. The lipid nanoparticle of any one of claims 1-37, wherein the nucleic acid sequence is a RNA sequence.

40. A pharmaceutical composition, comprising: (a) the lipid nanoparticle according to any one of claims 1-39, and (b) a pharmaceutically acceptable excipient.

41. A method of disrupting the transcription of an exon of an HIV-1 sequence in an individual in need thereof, comprising administering to the individual the lipid nanoparticle according to any one of claims 1-39 or the composition of claim 40.

42. A method of excising all or a portion of an HIV-1 sequence in an individual in need thereof, comprising administering to the individual the lipid nanoparticle according to any one of claims 1-39 or the composition of claim 40.

43. A method of treating an HIV-1 infection in an individual in need thereof, comprising administering to the individual the lipid nanoparticle according to any one of claims 1-39 or the composition of claim 40.

44. A method of preventing an HIV-1 infection in an individual in need thereof, comprising prophylactically administering to the individual the lipid nanoparticle according to any one of claims 1-39 or the composition of claim 40.

45. A method of preventing transmission of an HIV-1 virus from a first individual to a second individual, comprising administering to the first individual the lipid nanoparticle according to any one of claims 1-39 or the composition of claim 40.

46. The method of claim 45, wherein the first individual is a pregnant woman and the second individual is a child.
